(19) United States
(12) Patent Application Publication
(10) Pub. No.: US 2022/0142576 A1
(43) Pub. Date: May 12, 2022

(54) METHOD OF EXTRACTING PHYSIOLOGICAL DATA FROM A WEARABALE PHYSIOLOGICAL DATA ACQUIRER AND DOCKING STATION THEREFORE

(71) Applicant: ICENTIA INC., Québec (CA)

(72) Inventors: Pierre PAQUET, Quebec (CA); Yannick LE DEVEHAT, Québec (CA)

(21) Appl. No.: 17/438,522

(22) PCT Filed: Mar. 16, 2020
(86) PCT No.: PCT/CA2020/050349

§ 371 Date: Sep. 13, 2021

Related U.S. Application Data
(60) Provisional application No. 62/821,906, filed on Mar. 21, 2019.

(51) Int. Cl. A61B 5/00; G16H 40/67; G06F 1/16

(52) U.S. Cl. CPC A61B 5/6823; G16H 40/67; A61B 2560/045; A61B 2560/0456; G06F 1/1632

(57) ABSTRACT

There is described a method of acquiring physiological data with a wearable physiological data acquirer positioned onto an upper body of a user. The method generally has: acquiring first physiological data with the wearable physiological acquirer worn on the upper body of the user; removing the wearable physiological data acquirer from the upper body of the user; docking the wearable the physiological data acquirer to a docking station using a wired connection; the docking station extracting the first physiological data from the wearable physiological data acquirer via the wired connection; un-docking the wearable physiological data acquirer from the docking station after the extracting; repositioning the wearable physiological acquirer onto the upper body of the user; and acquiring a second physiological data with the wearable physiological acquirer worn on the upper body of the user.

### FIELD

The improvements generally relate to methods and devices for obtaining physiological data such as electrocardiogram data from a user via a wearable physiological data acquirer.

### BACKGROUND

Traditionally, electrophysiological data, such as respiratory rate, heart rate or any other electrocardiogram data, were acquired from a user (typically a patient) by bulky devices (e.g., bed-side electrocardiography (ECG) monitors) at medical premises. These physiological data acquirers were generally suitable for acquiring physiological data over a relatively short period of time, typically no longer than a few hours, because it constrained the patient to the medical premises. They were ill suited, for instance, for acquiring sporadic or temporary conditions which do not necessarily occur during such a limited acquisition period. Recent developments have taken place in the field and a number of wearable physiological data acquirers have been developed (e.g., ambulatory Holter monitors).

One example is presented in published PCT application WO 2016/134473, where a physiological data acquirer has an integrated memory allowing in situ acquisition of the data. The physiological data acquirer can have a flexible bandage-like housing made of a water-resistant material and be provided with snap-button connectors adapted to snappingly receive electrodes. Accordingly, the physiological data can be recorded while allowing freedom of movement to the user. Once the acquisition period has ended, the physiological data acquirer can be sent to extraction premises where data extraction can take place via a data extraction device. The physiological data acquirer can be connected to the data extraction device via the snap-button connectors and the data can be extracted via the connected connectors. Subsequent advancements related to the above-identified technology are described in published PCT applications WO 2017/177298 and WO 2017/177299.

Such extraction techniques were found suitable to a certain degree, but there remained room for improvement. In particular, the existing technologies only provided medical personnel with access to the recorded data after the extraction stage, which necessarily followed the data acquisition stage and was further delayed by the transit and any extraction backlog at the extraction premises. In some cases, medical personnel may have preferred to have access to relevant events in the acquired data in a shorter period of time.

While published PCT application WO 2016/134473 presents the avenue of providing the physiological data acquirer with a transmitter to allow wireless transmission of the data, this latter avenue presents several inconveniences, including the fact that it requires power from the acquirer's battery, and can therefore either limit the acquisition period or increase the size of the battery.

### SUMMARY

A physiological data acquisition system is presented herein where a docking station is provided in combination with a wearable physiological data acquirer. The docking station can be conveniently located for the user, such as located at the user's home and can be powered by another power supply, such as being plugged into the home's electrical power supply. The user can wear electrodes adhered to the upper body, and the electrodes and acquirer can be provided with mating connectors which act both as electrical and mechanical connectors, via which the acquirer can be selectively connected to or disconnected from the electrodes. The docking station can also have connectors allowing its connection to the acquirer and be enabled to extract data from the acquirer via that wired connection. The extraction can be done using the docking station's power rather than the acquirer's power to preserve the acquirer's autonomy, which can be done by powering the acquirer data extraction (i.e., by using the same connectors). Accordingly, at any time during the acquisition period, the user can remove the acquirer by disconnecting it from the electrodes, dock it to the docking station where the data is extracted, un-dock it from the docking station and reposition it to the electrodes. The docking station can further be enabled for wired (e.g., ethernet) or wireless (e.g., wifi, mobile phone-type communication) communication, to allow transmission of the data. The docking station can also be pre-programmed to execute particular routines, for example extract certain amount of data at a given time. The docking station can also set or change parameters in the acquirer, for example to log the amount of data extracted or the time at which the extraction took place. The data can optionally be pre-processed by the acquirer, and/or by the docking station, before being transmitted by the docking station. The wearer can optionally dock the acquirer more than once during the acquisition period which can be for several days, for instance. With the docking station, the step of sending the wearable physiological data acquirer to a remote location for data extraction becomes optional, and it may become preferred to extract the data solely via the docking station. However, the wearable physiological data may nonetheless be sent to the extraction premises.

In accordance with a first aspect of the present disclosure, there is provided a method of acquiring physiological data with a wearable physiological data acquirer positioned onto an upper body of a user, the method comprising: acquiring first physiological data with the wearable physiological acquirer worn on the upper body of the user; removing the wearable physiological data acquirer from the upper body of the user; docking the wearable physiological data acquirer to a docking station using a wired connection; the docking station extracting the first physiological data from the wearable physiological data acquirer via the wired connection; un-docking the wearable physiological data acquirer from the docking station after the extracting; repositioning the wearable physiological acquirer onto the upper body of the user; and acquiring a second physiological data with the wearable physiological acquirer worn on the upper body of the user.

Further in accordance with the first aspect of the present disclosure, the wearable physiological acquirer can for example be powered by the docking station.

Still further in accordance with the first aspect of the present disclosure, the wearable physiological acquirer can for example be powered by the docking station during said extracting via a data extraction path.

Still further in accordance with the first aspect of the present disclosure, the user can for example perform the steps of removing, docking, un-docking and repositioning.

Still further in accordance with the first aspect of the present disclosure, the step of removing can for example include disconnecting connectors of the wearable physiological data acquirer from electrodes adhered to the upper body of the user.

Still further in accordance with the first aspect of the present disclosure, the step of docking can for example include connecting the connectors of the wearable physiological data acquirer to corresponding connectors of the docking station.

Still further in accordance with the first aspect of the present disclosure, the sequence can for example comprise said steps of acquiring, removing, docking, extracting, un-docking and repositioning being performed at least once within a predetermined acquisition period.

Still further in accordance with the first aspect of the present disclosure, the sequence can for example be performed a plurality of times within the predetermined acquisition period.

Still further in accordance with the first aspect of the present disclosure, the method can for example comprise, after said acquiring the second physiological data, a step of removing the wearable physiological data acquirer from the upper body of the user.

Still further in accordance with the first aspect of the present disclosure, the method can for example comprise a step of sending the wearable physiological data acquirer to a remote location.

Still further in accordance with the first aspect of the present disclosure, the docking station can for example transmit at least said first physiological data to a data treatment center.

Still further in accordance with the first aspect of the present disclosure, the step of extracting can for example be powered by the docking station.

Still further in accordance with the first aspect of the present disclosure, the method can for example comprise a step of pre-processing the first physiological data prior to said extracting.

In accordance with a second aspect of the present disclosure, there is provided a docking station for use with a wearable physiological data acquirer worn on an upper body of a user, the docking station comprising: a housing; a power supply unit; a computer being housed within the housing, the computer having a processor and a memory being communicatively coupled to one another and being powered by the power supply unit; and a data entry port being communicatively connected to the computer; the memory comprising instructions which when executed by the processor execute: upon the data entry port being connected to the wearable physiological data acquirer, and using power only from the power supply unit, extracting first physiological data from the wearable physiological acquirer.

Further in accordance with the second aspect of the present disclosure, the docking station can for example comprise a communication unit communicatively coupled to the computer, said communication unit being configured to transmit the first physiological data upon extraction.

Still further in accordance with the second aspect of the present disclosure, the first physiological data can for example be transmitted wirelessly.

In accordance with a third aspect of the present disclosure, there is provided a method of extracting physiological data from a wearable physiological data acquirer positioned onto an upper body of a user, the method comprising: acquiring first physiological data with the wearable physiological acquirer worn on the upper body of the user; while said wearable physiological data acquirer is positioned onto the upper body of the user: connecting the wearable physiological data acquirer to a docking station using a wired connection; the docking station extracting the first physiological data from said wearable physiological data acquirer via said wired connection; and un-docking said wearable physiological data acquirer from said docking station; acquiring second physiological data with the wearable physiological acquirer worn on the upper body of the user.

Many further features and combinations thereof concerning the present improvements will appear to those skilled in the art following a reading of the instant disclosure.

### DESCRIPTION OF THE FIGURES

In the figures,

FIG. 1 is a schematic view of an example of a physiological data acquisition system during use, in accordance with one or more embodiments;

FIG. 2 is a flowchart of an example of a method of acquiring physiological data with the physiological data acquisition system of FIG. 1, in accordance with one or more embodiments;

FIG. 3 is a schematic view of an example of a physiological data acquisition system, with a wearable physiological data acquirer having electrodes to be positioned onto the upper body of a user, in accordance with one or more embodiments;

FIG. 4 is a schematic view of an example of a physiological data acquisition system, with a wearable physiological data acquirer having snap-buttons to be snappingly received on electrodes positioned onto the upper body of a user, in accordance with one or more embodiments;

FIGS. 4A and 4B show a block diagram of the physiological data acquisition system of FIG. 4, in accordance with one or more embodiments; and

FIG. 5 is a schematic view of an example of a physiological data acquisition system, with a wearable physiological data acquirer being dockable to a docking station without being removed from the upper body of a user, in accordance with one or more embodiments.

### DETAILED DESCRIPTION

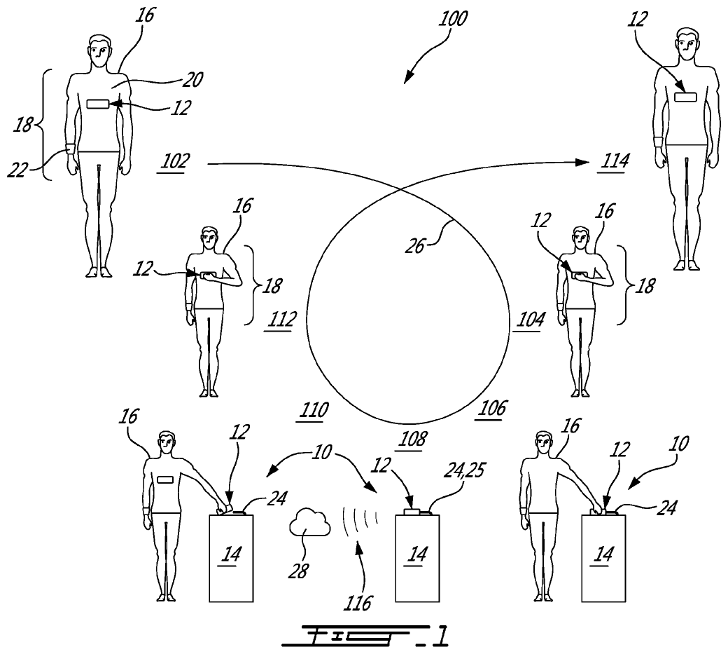

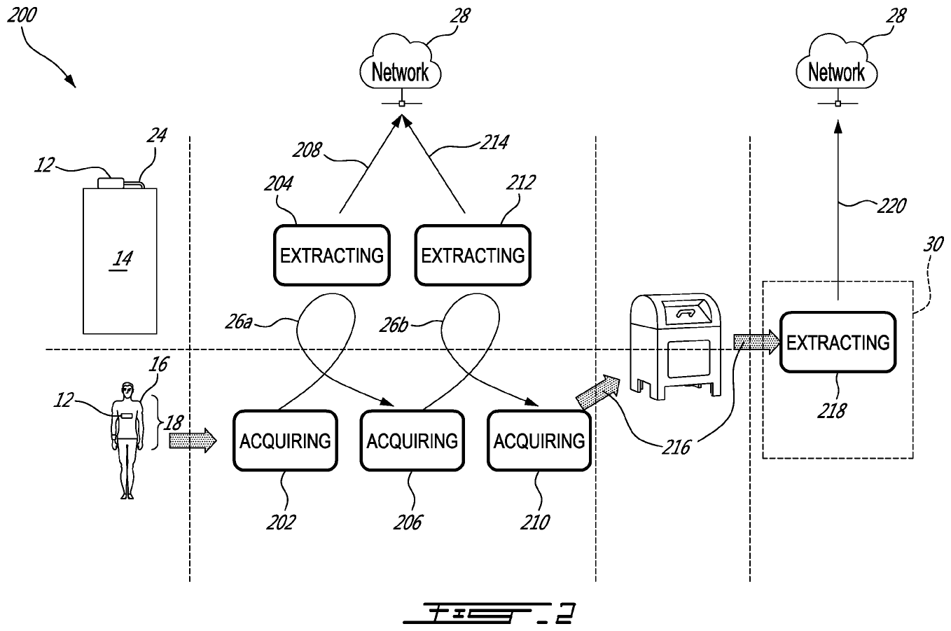

FIG. 1 shows various steps of an exemplary method 100 of acquiring physiological data with a physiological data acquisition system 10 comprising a wearable physiological data acquirer 12 (hereinafter “the acquirer 12”) and a docking station 14.

As will be understood, the acquirer 12 can be any type of electronic device which is configured for acquiring physiological data of a user 16 when worn on the upper body 18 of that user 16. Preferably, the acquirer 12 is suited to be worn on the torso 20. However, the acquirer 12 can be worn on the wrist 22 in some alternate embodiments.

It is noted that the physiological data should be acquired with sufficient resolution and precision so as to allow a skilled physician reviewing the physiological data to be able to diagnose common or uncommon health problems. For instance, in embodiments where the physiological data are electrocardiogram data, the skilled physician could diagnose heart problems including, but not limited to, heart failure, arrhythmia (e.g., tachycardia, bradycardia, premature ventricular contractions, and fibrillation), congenital heart disease, coronary artery disease, dilated cardiomyopathy, myocardial infarction, hypertrophic cardiomyopathy, mitral regurgitation, mitral valve prolapse, pulmonary stenosis and the like. Examples of such acquirers are described in PCT applications WO 2016/134473, WO 2017/177298 and WO 2017/177299, the contents of which are hereby incorporated by reference.

In the illustrated embodiment, the method 100 begins at step 102, where the acquirer 12 is worn on the upper body 18 of the user 16 for a first period of time during which first physiological data are acquired. At step 104, the acquirer 12 is removed from the upper body 18 of the user 16, thereby interrupting the acquisition of physiological data. The acquirer 12 is then docked to the docking station 14 using a wired connection 24 such as shown at step 106. At step 108, the previously acquired first physiological data are extracted from the docking station 14 by the docking station 14 after which, at step 110, the acquirer 12 is un-docked from the docking station 14 to be repositioned onto the upper body 18 of the user 16 at step 112. Once the acquirer 12 is so-repositioned, second physiological data are acquired for a second period of time such as shown at step 114. In some embodiments, the acquirer 12 can be powered by the docking station 14. For instance, the docking station 14 may power the acquirer 12 prior, during or after the data extraction. In these embodiments, the acquirer 12 can be powered by the docking station 14 via a data extraction path 25.

As shown, the method 100 has a data extraction loop 26 which can be initiated at any time during the acquisition of the prior, first physiological data, during step 102, and which can end back in the acquisition of the subsequent, second physiological data, at step 114. In some embodiments, extraction can start automatically as the acquirer 12 is placed on the docking station 14. Data acquisition can resume automatically as soon as the acquirer 12 is repositioned on the upper body 18 of the user 16. More specifically, the data extraction loop 26 comprises the step 104 of removing, the step 106 of docking, the step 108 of extracting, the step of 110 of un-docking and the step 112 of repositioning. When docked, the acquirer 12 can be powered by the docking station 14.

Accordingly, in embodiments where a skilled physician has prescribed the use of the acquirer 12 to the user 16, and thereby to acquire physiological data during a predetermined amount of time (hereinafter referred to as “predetermined acquisition period”), the data extraction loop 26 can be performed at least once, or preferably many times, within the predetermined acquisition period.

In an example situation, if the predetermined acquisition period is one week, then the data extraction loop 26 can be performed daily. In this situation, first physiological data are extracted after a first day of acquisition, second physiological data are extracted after a second day of acquisition, and so forth, until the expiry of the predetermined acquisition period.

After the predetermined acquisition period, the acquirer 12 may be disposed of, or alternately sent to a remote location for recycling, re-use of certain parts, and/or further data extraction. Examples of such remote location can include, but not limited to, extraction premises, recycling premises, and/or medical premises.

Preferably, the docking station 14 is configured to transmit the physiological data to a remote location, and will typically do so upon each data extraction loop 26, or shortly afterwards, such as shown at step 116. In some embodiments, the physiological data can be transmitted to the remote location directly. In some other embodiments, the physiological data can be transmitted to the remote location indirectly via a network 28. The docking station can be programmed remotely to transfer its data on predetermined time intervals or at specific times. The communication between the docking station 14 and the network 28 can be wired, wireless, or a combination thereof. As can be understood, examples of the network 28 can include the Internet, an intranet or any other suitable network by which data can be transmitted and/or received.

In the example situation discussed above, if a skilled physician has access to the network 28 and/or to the remote location where the physiological data are transmitted, the skilled physician can review the physiological data of the user 16 as they are received from the docking station 14, once or many times prior to the expiry of the predetermined acquisition period.

The inventors found that the method 100 and system 10 described herein could allow the skilled physician to detect abnormalities in the physiological data, if any, and react accordingly even before the expiry of the predetermined acquisition period, to the benefit of the user's health, in contrast with existing physiological data acquisition systems which allowed data extraction and review only after the entirety of the predetermined acquisition period had elapsed.

Moreover, as will be discussed below, in some embodiments, the docking station 14 is configured so that the step 108 of extracting can draw power only from the docking station 14 itself, and not from the acquirer 12. The inventors found this feature to be convenient as it can extend the battery life of the acquirer 12, thus allowing it to acquire physiological data for a longer period of time, if need be.

It is noted that in the illustrated embodiment manipulations during the data extraction loop 26 are performed by the user 16. However, in some other embodiments, these manipulations can be performed by a third party person such as a nurse, a caregiver or a family member, should the user be inexperienced or incapacitated in any way. Of course, one or more of the steps 104, 106, 110 and 112 can be performed by the user 16 whereas the remaining one(s) of the steps 104, 106, 110 and 112 can be performed by the third party person, in some alternate embodiments.

Depending on the embodiment, the physiological data extracted from the acquirer 12 are either raw physiological data or pre-processed physiological data. In the latter embodiment, the acquirer 12 can include a processor configured for pre-processing the acquired physiological data according to predetermined algorithm(s) stored on a memory of the acquirer 12 and/or programmed into the acquirer 12 by the docking station 14 at the time of extraction. Alternately or additionally, the physiological data transmitted from the docking station 14 are either raw or pre-processed. In the latter embodiment, the docking station 14 can include a processor configured for pre-processing the extracted physiological data according to predetermined algorithms stored on a memory of the docking station 14.

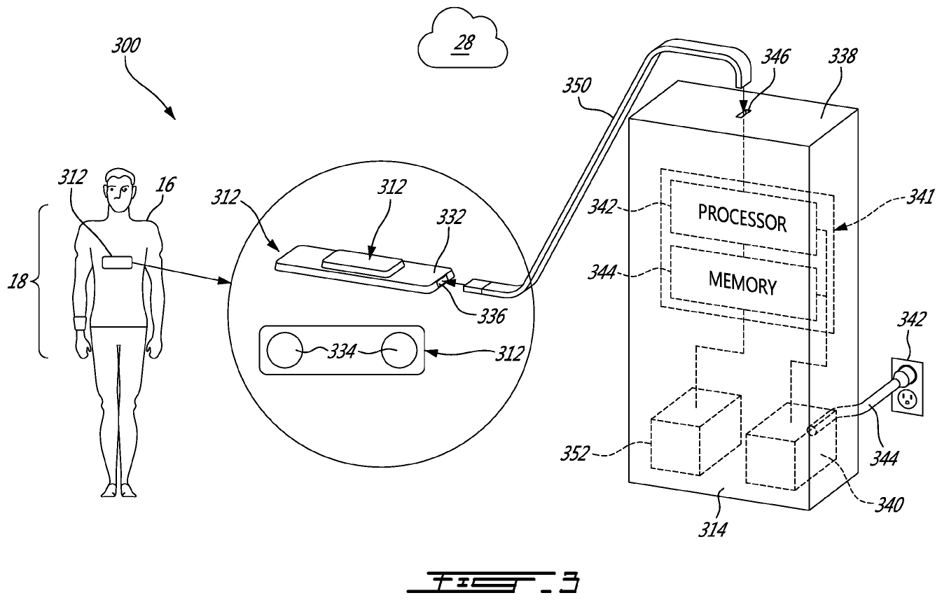

FIG. 2 shows a flowchart of an example of a method 200 of acquiring physiological data with the physiological data acquisition system 10 described above with reference to FIG. 1. More specifically, the flowchart represents a time line, extending from left to right, schematically showing its steps as they are sequentially performed.

At 202, the acquirer 12 is positioned onto the upper body 18 of the user 16, where first physiological data are acquired. For the sake of brevity, the predetermined acquisition period in this example has been set up to three days, with a data extraction loop to be performed daily.

As such, a first data extraction loop 26a is performed during which the acquirer 12 is removed from the upper body 18 of the user 16. The acquirer 12 is then docked onto the docking station 14 which extracts the first physiological data from the acquirer 12 at 204. The acquirer 12 is then repositioned on the upper body 18 of the user 16, after which the acquirer 12 acquires subsequent, second physiological data such as shown at 206. After extraction of the first physiological data from the acquirer 12 at 204, the first physiological data are transmitted to the network 28 at 208 where they may be accessible and/or reviewed by the skilled physician.

A second data extraction loop 26b is then performed, during which the second physiological data are extracted from the acquirer 12, and which ultimately leads to the acquirer acquiring subsequent, third physiological data at 210. Again, after extraction of the second physiological data from the acquirer 12 at 212, the second physiological data are also transmitted to the network 28 at 214.

As shown in this embodiment, the acquisition of the third physiological data is interrupted prior to a third data extraction loop (not shown). In this case, the acquirer 12 may be sent at step 216, for instance via mail to a remote location 30 where the third physiological data may be extracted at 218 and then transmitted to the network 28 as well, at 220. As can be understood, after the extraction of the third physiological data, the acquirer 12 can be disposed of, or alternately used for recycling and/or re-use of certain parts.

In some other embodiments, however, a third data extraction loop could have been performed to extract the third physiological data from the acquirer 12 using the docking station 14, after which the third physiological data could have been sent to the network 28. In which case, instead of sending the acquirer 12 to the remote location 30, the acquirer could have been disposed of.

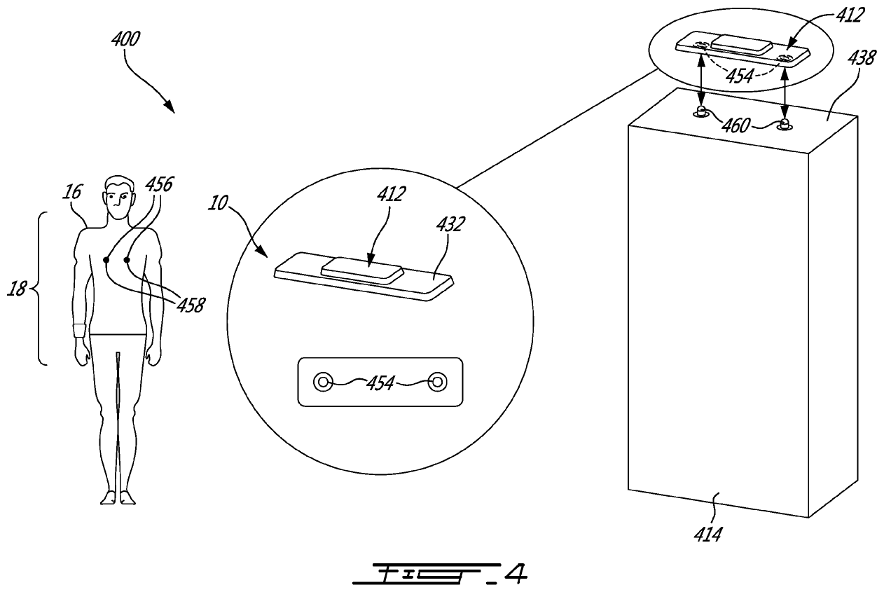

FIG. 3 shows another example of a data acquisition system 300 having an acquirer 312 and a docking station 314. As shown in this example, the acquirer 312 has a flexible bandage-like housing 332 and is provided with electrodes 334 thereunder which can be adhered to the upper body 18 of the user 16 so as to acquire physiological data therefrom. As shown, the housing 332 of the acquirer 312 has a data exit port 336.

As depicted, the docking station 314 has a housing 338, a power supply unit 340, a computer 341 which is housed within the housing 338. The computer 341 has a processor 342 and a memory 344 being communicatively coupled to one another and which are both powered by the power supply unit 340. In this example, the power supply unit 340 is connected to a power outlet 342 in the home of the user 16 via a power cable 344. However, the power supply unit 340 could have alternately been provided in the form of a battery or battery pack in other embodiments. As shown, the docking station 314 has a data entry port 346 to which the acquirer 312 is connected via at least one data wire 350 when the acquirer 312 is docked to the docking station 314.

As can be understood, the memory 344 comprises instructions which when executed by the processor 342 executes the step of extracting previously acquired physiological data from the acquirer 312, upon connection between the data entry port 346 of the docking station 314 and the data exit port 336 of the acquirer 312, and using power only from the power supply unit 340. In some embodiments, the docking station 314 can have one or more buttons to initiate the extraction step. However, these buttons can be omitted.

In this specific embodiment, the docking station 314 can have a communication unit 352 which is communicatively coupled to the computer 341 and to the network 28. Accordingly, the communication unit 352 is configured to transmit the physiological data to the network 28 upon extraction, or shortly afterwards. In this embodiment, the connection between the communication unit 352 and the network 28 is wireless.

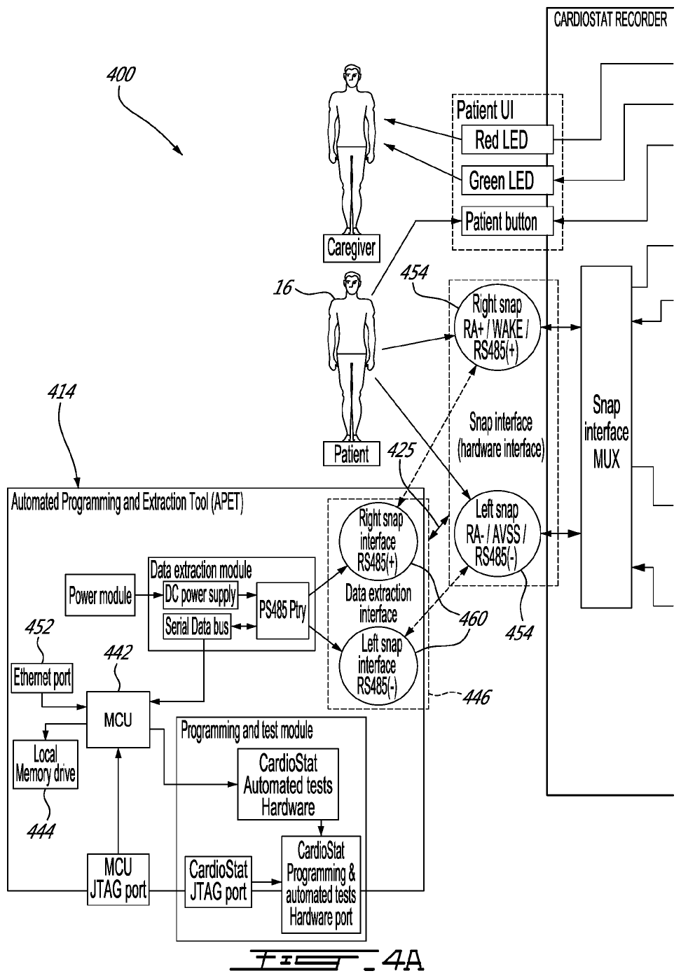

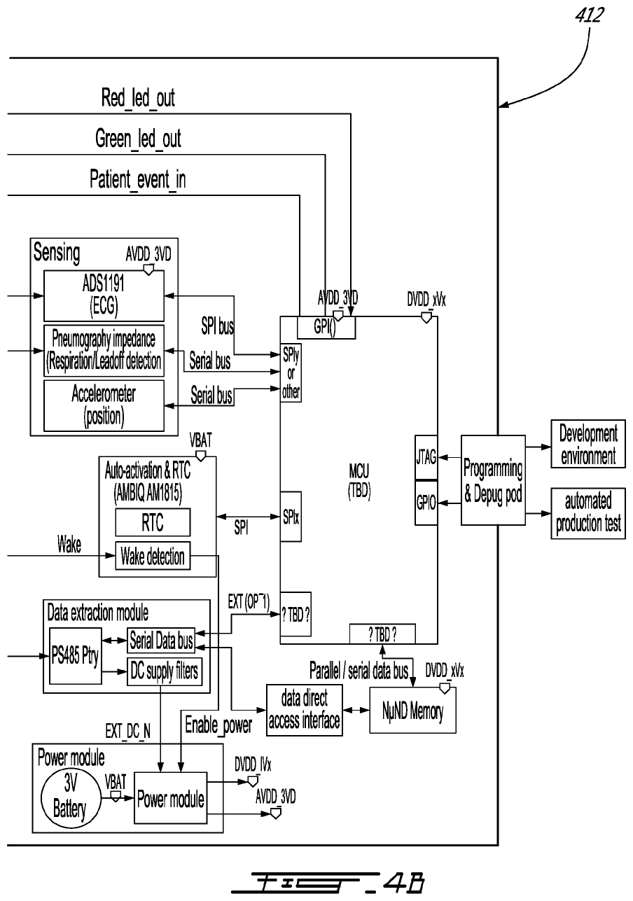

FIG. 4 shows an example of a physiological data acquisition system 400 having an acquirer 412 and a docking station 414. As depicted, the acquirer 412 has a flexible bandage-like housing 432 made of a water-resistant material and is provided with snap-button connectors 454 adapted to snappingly receive electrodes 456 being adhered to the upper body 18 of the user 16. In this example, the electrodes 456 have protruding male snap-buttons which can be snappingly received corresponding female snap-buttons of the acquirer 412.

In this example, the step of removing the acquirer 412 from the upper body 18 of the user 16 can include a step of disconnecting the female snap-buttons or otherwise similar connectors 454 of the acquirer 412 from the male snap-buttons or otherwise similar connectors 458 of the electrodes 456 adhered to the upper body 18 of the user 16. Similarly, the step of docking the acquirer 412 to the docking station 414 can include a step of connecting the female-snap-buttons or otherwise similar connectors 454 of the acquirer 412 to male snap-buttons or otherwise similar connectors 460 of the docking station 414. As mentioned above, power can be drawn from the docking station 414 towards the acquirer 412 to power and/or charge it while they are connected to one another via the connectors 454 and 460. Accordingly, power can be transferred between the acquirer 412 and the docking station 414 while data are exchanged between the acquirer 412 and the docking station 414. In some embodiments, the power and the data can be exchanged using a shared, common data extraction path 425 extending between the connectors 454 and 460.

As shown, the docking station 414 has a housing 438 with protruding male snap-button connectors 460 similar to the ones of the electrodes 456 to which the female snap-buttons 454 of the acquirer 412 can be connected, thus forming a wired connection. As discussed in PCT published application WO 2016/134473, the data extraction can be performed via the connected connectors 454 and 460. Such connectors have been found to be satisfactory, especially as they can allow the acquirer 412 to remain watertight due to the absence of other data exit ports thereon. It can also simplify the data extraction process as no further manipulation may be needed to be done on the acquirer 412 in order to extract the data. In some embodiments, such manipulation can include opening the housing to take the memory, and/or other electronic components, out of the housing.

FIGS. 4A and 4B show a detailed block diagram of the physiological data acquisition system 400. As shown, the snap-button connectors 454 have a right snap RS485(+) and a left snap RS485(−) which collectively form the data entry port 446. The processor 442 is provided in the form of a microcontroller unit, the memory 444 is provided in the form of a local memory drive, the communication unit 452 is provided in the form of an ethernet port, the power supply unit 414 is provided in the form of a power module and DC power supply. The computer includes a RS485 interface circuit and a serial data bus. The docking station 414 can also be provided with a programming and test module for programming the extraction of the data out of the acquirer 412 and performing tests as desired.

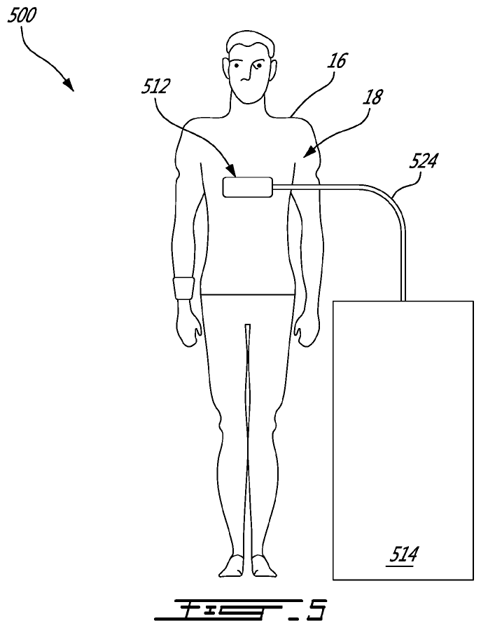

FIG. 5 shows another example of a physiological data acquisition system 500. As depicted, a data extraction loop can be performed all the while the acquirer 512 is still positioned onto the upper body 18 of the user 16. More specifically, first physiological data are acquired with the acquirer 512 worn on the upper body 18 of the user 16. Then, while the acquirer 512 stays positioned on the upper body 18 of the user 16, the acquirer 512 is connected to the docking station 514 using a wired connection 524, whereby the docking station 524 extracts the first physiological data from the acquirer 512. The acquirer 512 can be un-docked from the docking station 514 by disconnecting the wired connection 524 therebetween, thereby allowing the acquirer 512 to acquire subsequent, second physiological data.

As can be understood, the examples described above and illustrated are intended to be exemplary only. For instance, although the figures show the acquirer being positioned onto the thorax of the user, the acquirer can alternately be positioned onto the abdomen, arms or other suitable parts of the user. It is intended that upon extraction of some physiological data, the acquirer and/or the docking station can be configured to annotate and/or recognize the physiological data that have already been extracted, and/or to delete the extracted physiological data from the memory of the acquirer. Moreover, the acquirer and/or the docking station can be configured to annotate and/or recognize the physiological data which have been extracted, to avoid extracting the same physiological data twice, for instance. Of course, the physiological data can be timestamped so as to allow the skilled physician to match a one or more portions of the physiological data with one or more real-world events should it be necessary in some applications. The scope is indicated by the appended claims.

What is claimed is:

1. A method of acquiring physiological data with a wearable physiological data acquirer positioned onto an upper body of a user, the method comprising:
acquiring first physiological data with the wearable physiological acquirer worn on the upper body of the user;
removing the wearable physiological data acquirer from the upper body of the user;
docking the wearable physiological data acquirer to a docking station using a wired connection;
the docking station extracting the first physiological data from the wearable physiological data acquirer via the wired connection;
un-docking the wearable physiological data acquirer from the docking station after the extracting;
repositioning the wearable physiological acquirer onto the upper body of the user; and
acquiring a second physiological data with the wearable physiological acquirer worn on the upper body of the user.

2. The method of claim 1 wherein the wearable physiological acquirer is powered by the docking station via said wired connection.

3. The method of claim 2 wherein the wearable physiological acquirer is powered by the docking station during said extracting via a data extraction path of said wired connection.

4. The method of claim 1 wherein the user performs said steps of removing, docking, un-docking and repositioning.

5. The method of claim 1 wherein said removing includes disconnecting connectors of the wearable physiological data acquirer from electrodes adhered to the upper body of the user.

6. The method of claim 5 wherein said docking includes connecting the connectors of the wearable physiological data acquirer to corresponding connectors of the docking station.

7. The method of claim 1 wherein a sequence comprising said steps of acquiring, removing, docking, extracting, un-docking and repositioning being performed at least once within a predetermined acquisition period.

8. The method of claim 7 wherein said sequence is performed a plurality of times within the predetermined acquisition period.

9. The method of claim 1 further comprising, after said acquiring the second physiological data, removing the wearable physiological data acquirer from the upper body of the user.

10. The method of claim 9 further comprising sending the wearable physiological data acquirer to a remote location.

11. The method of claim 1 wherein the docking station transmits at least said first physiological data to a data treatment center.

12. The method of claim 1 wherein said extracting is powered by the docking station.

13. The method of claim 1 further comprising pre-processing the first physiological data prior to said extracting.

14. A docking station for use with a wearable physiological data acquirer worn on an upper body of a user, the docking station comprising:
a housing;
a power supply unit;
a computer being housed within the housing, the computer having a processor and a memory being communicatively coupled to one another and being powered by the power supply unit; and
a data entry port being communicatively connected to the computer;
the memory comprising instructions which when executed by the processor execute:
upon the data entry port being connected to the wearable physiological data acquirer, and using power only from the power supply unit, extracting first physiological data from the wearable physiological acquirer.

15. The docking station of claim 14 further comprising a communication unit communicatively coupled to the computer, said communication unit being configured to transmit the first physiological data upon extraction.

16. The docking station of claim 15 wherein said first physiological data is transmitted wirelessly.

17. A method of extracting physiological data from a wearable physiological data acquirer positioned onto an upper body of a user, the method comprising:
acquiring first physiological data with the wearable physiological acquirer worn on the upper body of the user;
while said wearable physiological data acquirer is positioned onto the upper body of the user:
connecting the wearable physiological data acquirer to a docking station using a wired connection;
the docking station extracting the first physiological data from said wearable physiological data acquirer via said wired connection; and
un-docking said wearable physiological data acquirer from said docking station;
acquiring second physiological data with the wearable physiological acquirer worn on the upper body of the user.
